(19) United States
(12) Patent Application Publication
(10) Pub. No.: US 2025/0108169 A1
(43) Pub. Date: Apr. 3, 2025

(54) Feedback mechanism

(71) Applicant: SHL Medical AG, Zug (CH)

(72) Inventor: Jason Mondro, Deerfield Beach, FL (US)

(21) Appl. No.: 18/729,946

(22) PCT Filed: Jan. 9, 2023
(86) PCT No.: PCT/EP2023/050276

§ 371 Date: Jul. 18, 2024

Related U.S. Application Data
(60) Provisional application No. 63/301,516, filed on Jan. 21, 2022.

(30) Foreign Application Priority Data
Feb. 2, 2022 (EP) 22154707.8

(51) Int. Cl. A61M 5/315; A61M 5/50

(52) U.S. Cl. CPC A61M 5/31511; A61M 5/5066

(57) ABSTRACT

A feedback mechanism (300) for a medicament delivery device, the feedback mechanism (300) comprising a feedback portion (30) and a plunger rod (12): wherein the plunger rod (12) comprises an engagement structure (121): wherein the feedback portion (30) comprises one or more attachment parts (32); wherein the one or more attachment parts (32) are configured to connect the feedback portion (30) to another part of the medicament delivery device: wherein the feedback portion (30) is configured to engage with the engagement structure (121) during use of the medicament delivery device so that the one or more attachment parts (32) detach or break during movement of the plunger rod (12) relative to the feedback portion (30).

### CROSS-REFERENCE TO RELATED APPLICATIONS

The present application is a U.S. National Phase Application pursuant to 35 U.S.C. § 371 of International Application No. PCT/EP2023/050276 filed Jan. 9, 2023, which claims priority to (i) U.S. Provisional Patent Application No. 63/301,516 filed Jan. 21, 2022, and (ii) European Patent Application No. 22154707.8 filed Feb. 2, 2022. The entire disclosure contents of these applications are herewith incorporated by reference into the present application.

### TECHNICAL FIELD

The present disclosure relates to feedback mechanisms for medicament delivery devices and in to feedback mechanisms with a part that detaches or break during movement of a plunger rod.

### BACKGROUND

Some medicament delivery devices provide feedback to their users during operation, such as with the U-bracket described in WO2011/123024. While such systems can provide effective feedback, the applicant has appreciated that an alternative feedback mechanism could usefully be provided, potentially providing various advantages when compared to existing feedback mechanisms.

### BRIEF DESCRIPTION

A first aspect concerns a feedback mechanism for a medicament delivery device, the feedback mechanism comprising a feedback portion and a plunger rod. The plunger rod comprises an engagement structure. The feedback portion comprises one or more attachment parts. The one or more attachment parts are configured to connect the feedback portion to another part of the medicament delivery device. The feedback portion is configured to engage with the engagement structure during use of the medicament delivery device so that the one or more attachment parts detach or break during movement of the plunger rod relative to the feedback portion. This can provide a feedback mechanism, for example at the beginning or at the end of medicament delivery.

Optionally, the one or more attachment parts are configured to connect the feedback portion to a rear subassembly of the medicament delivery device.

Optionally, the feedback portion comprises a central opening through which the plunger rod can move towards a proximal end of the medicament delivery device.

Optionally, the engagement structure is attached to the plunger rod at a distal end of the plunger rod. This can allow feedback to be generated at the end of the injection, i.e. after the medicament is expelled from the container.

Optionally, the engagement structure is attached to the plunger rod at a proximal end of the plunger rod. This can allow feedback to be generated at the beginning of the injection, i.e. before the medicament is expelled from the container.

Optionally, after the engagement of the plunger rod with the feedback portion a displacement in proximal direction of the plunger rod causes a breakage of the one or more attachment parts.

Optionally, when the engagement structure is attached at the distal end of the plunger rod to indicate an end of an injection of a dose of a medicament; or is attached at the proximal end of the plunger rod to indicate a beginning of an injection of a dose of medicament.

Optionally, the feedback portion and the one or more attachment parts are moulded as one piece with a rear cap of the rear subassembly. This can minimise the number of parts in a medicament delivery device and/or reduce the number of assembly step needed during assembly.

Optionally, the feedback portion is welded or glued to the one or more attachment parts, and/or the one or more attachment parts are welded or glued to the rear subassembly. A potential advantage of gluing or welding additional pieces to the rear assembly is that previously existing pieces or moulds can be used for this new function.

Optionally, the one or more attachment parts are configured to comprise a breaking point such that the force of a spring that displaces the plunger rod is sufficient for breaking off all of the one or more attachment parts from the rear subassembly.

Optionally, the one or more attachment parts are configured to comprise a breaking point such that the force of a spring that displaces the plunger rod is sufficient for breaking off all of the one or more attachment parts from a rear cap of the rear subassembly or from the housing.

Optionally, the one or more attachment parts comprises a material and/or shape such that the material or shape allows a breaking of the one or more attachment parts when the force of the spring is applied.

Optionally, the feedback portion is a circular disc.

A second aspect concerns a medicament delivery device comprising the feedback mechanism according to any one of the previous options.

Optionally, the medicament delivery device further comprises: a tubular housing; a front subassembly, the front subassembly comprising a medicament container and a medicament delivery member; and a rear subassembly comprising an activation mechanism and an actuation mechanism for expelling a medicament, wherein the actuation mechanism is arranged to cause the plunger rod to expel a medicament out of the medicament container. Optionally, the actuation mechanism comprises a spring, a gas canister, or an electric motor.

The present disclosure is applicable to a number of medical delivery devices, including, but not limited to, devices that automatically, semi-automatically or manually deliver a dose of medicament through injection (needle and needleless).

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component. The circumferential direction describes a direction extending around the axis, so around the circumference of a device or component, and the radial direction extends perpendicular to the axis. Similarly, the terms “transverse”, “transversal” and “transversally” refer to a direction generally perpendicular to the longitudinal direction.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

The term automatic in conjunction with a medical delivery device means that the medicament delivery device is able to perform the injection without the user of the device delivering the force needed to expel the medicament during delivery. The force is delivered automatically by a resilient member like a spring or an electric motor or any other suitable means for providing the delivery force.

These and other aspects and advantages of the present invention will become apparent from the following detailed description and from the accompanying drawings.

### BRIEF DESCRIPTION OF THE DRAWINGS

Embodiments of the invention will now be described by way of example only and with reference to the accompanying drawings, in which:

FIG. 1 shows a perspective view of a portion of a medicament delivery device with a feedback mechanism in an initial state.

FIG. 2 shows a perspective view of part of the rear portion of the medicament delivery device, comprising the feedback mechanism in an initial state.

FIG. 3 shows a perspective view of the portion of the medicament delivery device of FIG. 1 with the feedback mechanism in a final state.

FIG. 4 shows a perspective view of part of the medicament delivery device.

FIG. 5 shows the feedback mechanism with the plunger rod with the engagement structure.

FIG. 6 shows a medicament delivery device having a tubular housing.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the figures.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

As used herein, apparatus, element and method “configured to” perform a specified function is indeed capable of performing the specified function without any alteration, rather than merely having potential to perform the specified function after further modification. In other words, the apparatus, element, and method “configured to” perform a specified function is specifically selected, created, implemented, utilized, programmed, and/or designed for the purpose of performing the specified function. As used herein, “configured to” refers to existing characteristics of an apparatus, element, and method which enable the apparatus, element, and method to perform the specified function without further modification. For purposes of this disclosure, an apparatus, element, and method described as being “configured to” perform a particular function can additionally or alternatively be described as being “adapted to” and/or as being “operative to” perform that function.

Further, in the following description, the wording medicament delivery device will be used. In this context, the term medicament delivery device may include various devices capable of delivering certain doses of medicament to a user. The medicament delivery devices may be either disposable or re-usable.

Although the medical device described below is a medicament delivery device configured as a disposable single-use, pen-type injector, such as an auto-injector, any other type of single-use automatic medicament delivery device could incorporate the feedback mechanisms of the present disclosure, including, but not limited to, inhalers or eye dispensers. Likewise, the medical device may be a training device that replicates a medicament delivery device.

A first aspect concerns a feedback mechanism 300 for a medicament delivery device, the feedback mechanism comprising a feedback portion 30 and a plunger rod 12. The plunger rod 12 comprises an engagement structure 121; wherein the feedback portion 30 comprises one or more attachment parts 32. The one or more attachment parts 12 are configured to connect the feedback portion 30 to another part of the medicament delivery device; wherein the feedback portion 30 is configured to engage with the engagement structure 121 during use of the medicament delivery device so that the one or more attachment parts 32 detach or break during movement of the plunger rod 12 relative to the feedback portion 30.

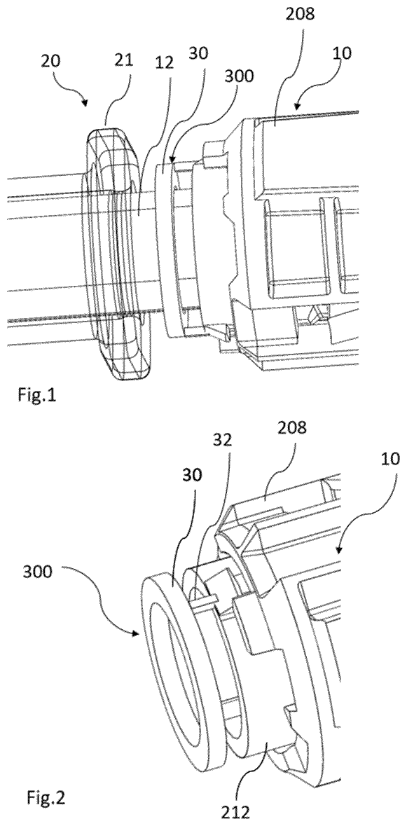

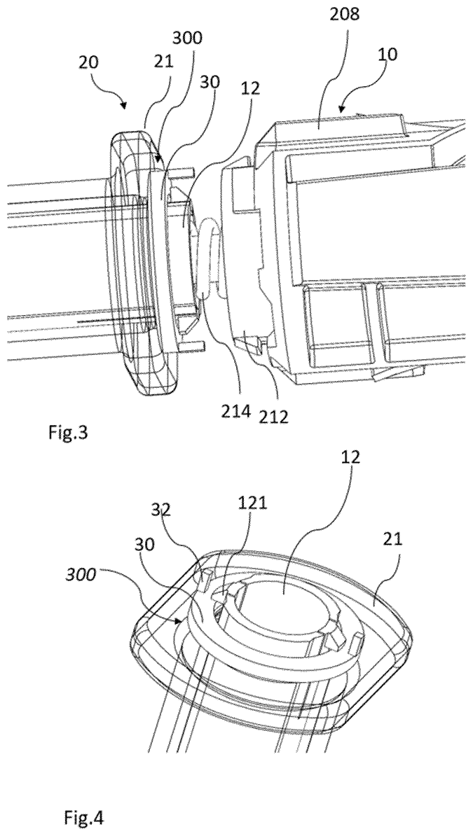

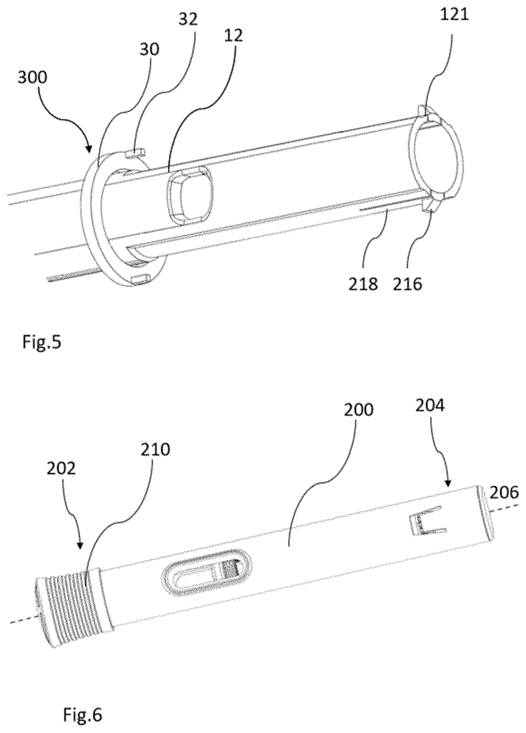

An example feedback mechanism is depicted in FIGS. 1 and 2, and will be described in more detail below. The feedback mechanism 300 as shown in FIGS. 1 and 2 can be implemented, for example, in an auto-injector, for example in an auto-injector designed and configured for automatically injecting a fixed dose of a medicament from a medicament container. The medicament container is typically positioned within a tubular housing of the medicament delivery device. The auto-injector can be configured as a single-use, disposable device. As shown in FIG. 6, the housing 200 has a proximal end 202, a distal end 204, and a longitudinal axis 206. Such a medicament delivery device is typically provided with a rear subassembly 10. The rear subassembly 10 typically comprises a rear cap 212, an activation mechanism, in this case a rotator 208 (partially visible in FIG. 1, for example), and/or a mechanism for expelling the medicament from the medicament container to the medicament delivery member. An example mechanism for expelling the medicament is a plunger rod 12 for expelling the medicament out of the medicament container 21. The plunger rod 12 can be actuated by an actuation mechanism for example a spring 214, gear wheels or threads. The plunger rod 12 typically has a tubular body extending from a distal end to a proximal end. The plunger rod 12 comprises an engagement structure 121 at the distal end of the plunger rod 12 (see FIG. 5). The engagement structure 121 in this example is a protrusion 216 extending circumferentially outwardly from the plunger rod body at the distal end of the plunger rod body. The protrusion 216 is at one end of an arm 218, with the other end of the arm 218 attached to the rest of the plunger rod, although the protrusion 216 could alternatively be attached directly to the rest of the plunger rod without any arm being provided. Alternatively, the engagement structure can be a hook element or flexible arms with protrusions extending circumferentially outwardly from the plunger rod body. The medicament delivery device further comprises a front subassembly 20 comprising the medicament container 21 and a medicament delivery member (not shown in the figures). Optionally, the front subassembly may comprise a cap (such as the cap 210 in FIG. 6) that, when attached, maintains the medicament delivery member (e.g. a needle) in a sterile condition. The rear subassembly 10 optionally comprises a rear cap 212 comprising an activation mechanism. In the example shown in the figures, a rear cap 212 is provided (see FIG. 2, for example), and the feedback portion 30 is attached to the rear cap.

In the present example, the feedback mechanism 300 comprises a feedback portion 30 as shown in FIGS. 1 and 2. In this example, the feedback portion 30 is a circular disc. Alternatively, the feedback portion 30 can be formed using other shapes; for example it can have a ring shape, a disc shape with a central opening, i.e. washer shape, or a half ring shape i.e. a C-shape. The feedback portion 30 comprises a central passage to receive the plunger rod 12. Further, the feedback mechanism 300 comprises one or more attachment elements 32. In the present example the attachment elements 32 have an elongated body for example in form of a thin rod or a thin plate. The attachment elements 32 extend from the rear cap 212 to the feedback portion 30. The attachment elements can alternatively be attached to a housing such as the housing 200 or to any other substructure of the rear assembly 10. The attachment parts 32 can be attached to a substructure already pre-existing within the activation mechanism of the rear subassembly, or an additional substructure could be added for this purpose.

The one or more attachment elements 32 are configured to connect the feedback portion 30 to the rear subassembly 10. In the depicted example, the feedback portion is attached to the rear cap 212.

In one example, the one or more attachment elements 32 are welded or glued to the feedback portion 30 and to the rear subassembly 10. Alternatively, the one or more attachment elements 32, the feedback portion 30 and the rear cap 212 are moulded as a single integral part. Similarly, where the feedback portion is attached to another component, such as to a housing, the feedback 30, the attachment elements and the component can be moulded as a single integral part.

In the present example, when the medicament delivery device is activated by a user, the activation mechanism is activated (in this by rotation of a rotator), allowing the expelling mechanism (a spring 214 in this example) to displace the plunger rod 12 towards the medicament container and the medicament delivery member, such that when the plunger rod reaches the end of dose, the distal part of the plunger rod 12 is configured to engage with the feedback portion 30. In other words, the engagement structure 121 of the plunger rod 12 engages with the feedback portion 30 during the proximal displacement of the plunger rod 12.

After the engagement of the plunger rod with the feedback portion 30, i.e. the engagement structure 121 engaging with the feedback portion 30, further displacement of the plunger rod 12 in proximal direction causes the breakage of the one or more attachment elements 32.

The breaking of the one or more attachment elements 32 causes an audible feedback in form of a clicking sound when breaking, thereby providing feedback. In the depicted example, the feedback is intended to indicate the end of dose delivery, although as discussed elsewhere, the feedback could be also arranged to provide a start of dose indication. Optionally, more than one feedback portion 30 could be provided, thereby allowing more than one click to be provided (one for each feedback portion), with clicks provided at the beginning (start), during, and/or at the end of a dose.

Optionally, subsequently to the breakage the feedback portion 30 is detached from the rear subassembly. Then the feedback portion 30 moves with the plunger rod 12 towards the medicament container 21, which can generate a second click when impacting a distal structure of the medicament container 21 (for example a syringe flange). This could allow a single feedback portion to provide two clicks.

Preferably, the one or more attachment elements 32 are configured to comprise a breaking point such that the force of the spring that displaces the plunger rod is sufficient to break the bond of all of the one or more attachment elements 32 from the rear subassembly 10.

The material and/or the shape of the one or more attachment elements 32 is such that it allows a breaking of the attachment element. For example, the attachment element 32 can have a triangular shape, i.e. a triangular plate, which might facilitate a breaking of the attachment element 32. In other words, one edge of the triangular plate is attached to the rear subassembly 10 and the two other edges are attached to the feedback portion 30.

Alternatively, the material and/or the shape of the one or more attachment elements 32 is such that it allows a detachment or release of the feedback portion 30. For example, the attachment elements have hook-like elastic end portions engaging with the rear subassembly or the feedback portion. The hook like elastic end portions are configured to keep the feedback portion attached to the rear subassembly and to release one of the attachments to the feedback portion or the rear sub assembly, such that the hook-like elastic end portions straighten under tension from the moving plunger rod 12, thereby disengaging the elastic hook-like end portions.

While various aspects and embodiments have been disclosed herein, other aspects and embodiments will be apparent to those skilled in the art. All embodiments within and between different aspects of the devices and methods can be combined unless the context clearly dictates otherwise. The various aspects and embodiments disclosed herein are for purposes of illustration and are not intended to be limiting, with the true scope and spirit being indicated by the claims.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-la′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Various modifications to the embodiments described are possible and will occur to those skilled in the art without departing from the invention which is defined by the following claims.

1-14. (canceled)

15. A feedback mechanism for a medicament delivery device, the feedback mechanism comprising a feedback portion and a plunger rod;
wherein the plunger rod comprises an engagement structure;
wherein the feedback portion comprises one or more attachment parts;
wherein the one or more attachment parts are configured to connect the feedback portion to another part of the medicament delivery device; and
wherein the feedback portion is configured to engage with the engagement structure during use of the medicament delivery device so that the one or more attachment parts detach or break during movement of the plunger rod relative to the feedback portion.

16. The feedback mechanism of claim 15, wherein the one or more attachment parts are configured to connect the feedback portion to a rear subassembly or to a housing of the medicament delivery device.

17. The feedback mechanism of claim 15, wherein the feedback portion comprises a central opening through which the plunger rod can move towards a proximal end of the medicament delivery device.

18. The feedback mechanism of claim 15, wherein the engagement structure is attached to the plunger rod at a distal end of the plunger rod.

19. The feedback mechanism of claim 15, wherein the engagement structure is attached to the plunger rod at a proximal end of the plunger rod.

20. The feedback mechanism of claim 18, wherein after the engagement of the plunger rod with the feedback portion, a displacement of the plunger rod in the proximal direction causes a breakage of the one or more attachment parts.

21. The feedback mechanism of claim 20, wherein when the engagement structure is attached at the distal end of the plunger rod to indicate an end of an injection of a dose of a medicament; or is attached at the proximal end of the plunger rod to indicate a beginning of an injection of a dose of medicament.

22. The feedback mechanism of claim 15, wherein the feedback portion, the one or more attachment parts and a rear cap of the rear subassembly are moulded as one piece.

23. The feedback mechanism of claim 15, wherein the feedback portion is welded or glued to the one or more attachment parts, and/or the one or more attachment parts are welded or glued to the rear subassembly or to the housing.

24. The feedback mechanism of claim 15, wherein the one or more attachment parts are configured to comprise a breaking point such that the force of a spring that displaces the plunger rod is sufficient for breaking off all of the one or more attachment parts from a rear cap of the rear subassembly or from the housing.

25. The feedback mechanism of claim 15, wherein the feedback portion is a circular disc.

26. A medicament delivery device comprising the feedback mechanism according to claim 15.

27. The medicament delivery device of claim 26, further comprising:
a tubular housing;
a front subassembly, the front subassembly comprising a medicament container and a medicament delivery member; and
a rear subassembly comprising an activation mechanism and an actuation mechanism for expelling a medicament, wherein the actuation mechanism is arranged to cause the plunger rod to expel a medicament out of the medicament container.

28. The medicament delivery device of claim 26, wherein the medicament delivery device is a single-use disposable device.
